(19) United States
(12) Patent Application Publication
(10) Pub. No.: US 2012/0316365 A1
(43) Pub. Date: Dec. 13, 2012

(54) Dehydrogenation Process

(76) Inventors: Teng Xu, Houston, TX (US); Edward A. Lemon, JR., Easton, PA (US); Wenyih F. Lai, Bridgewater, NJ (US); George H. Gamble, Long Valley, NJ (US)

(21) Appl. No.: 13/511,453

(22) PCT Filed: Dec. 17, 2010
(86) PCT No.: PCT/US2010/061012

§ 371 Date: Aug. 17, 2012

Related U.S. Application Data
(60) Provisional application No. 61/301,708, filed on Feb. 5, 2010.

(51) Int. Cl. C07C 45/61; C07C 5/367; C07C 15/04; C07C 49/543

(52) U.S. Cl. 568/343; 585/434

(57) ABSTRACT

In a process for the dehydrogenation of cyclohexanone to produce phenol, a feed comprising cyclohexanone is contacted with a catalyst comprising an inorganic, crystalline, mesoporous support material and a hydrogenation-dehydrogenation component under dehydrogenation conditions effective to convert at least part of the cyclohexanone in the feed into phenol and hydrogen.

### PRIORITY CLAIM

This application claims priority to U.S. Provisional Application Ser. No. 61/301,780 filed Feb. 5, 2010, the disclosure of which is fully incorporated herein by reference.

### CROSS REFERENCE TO RELATED APPLICATIONS

This patent application is related to U.S. Provisional Application Ser. No. 61/301,786, filed Feb. 5, 2010; U.S. Provisional Application Ser. No. 61/301,794 filed Feb. 5, 2010; and U.S. Provisional Application Ser. No. 61/301,799 filed Feb. 5, 2010, the disclosures of which are fully incorporated herein by their reference.

### FIELD

The present invention relates to a dehydrogenation process, specifically optimum catalyst compositions for the dehydrogenation of a dehydrogenatable hydrocarbon such as cyclohexanone.

### BACKGROUND

Various dehydrogenation processes have been proposed to dehydrogenate dehydrogenatable hydrocarbons such as cyclohexanone and cyclohexane. For example, these dehydrogenation processes have been used to convert at least a portion of the cyclohexanone into phenol.

Phenol is an important product in the chemical industry and is useful in, for example, the production of phenolic resins, bisphenol A, ε-caprolactam, adipic acid, and plasticizers.

Currently, the most common route for the production of phenol is the Hock process. This is a three-step process in which the first step involves alkylation of benzene with propylene to produce cumene, followed by oxidation of the cumene to the corresponding hydroperoxide and then cleavage of the hydroperoxide to produce equimolar amounts of phenol and acetone.

Other known routes for the production of phenol involve the direct oxidation of benzene, the oxidation of toluene, and the oxidation of s-butylbenzene wherein methyl ethyl ketone is co-produced with phenol in lieu of acetone produced in the Hock process.

Additionally, phenol can be produced by the oxidation of cyclohexylbenzene to cyclohexylbenzene hydroperoxide wherein cyclohexanone is co-produced with phenol in lieu of acetone produced in the Hock process. A producer using this process may desire to dehydrogenate at least a portion of the cyclohexanone produced into the additional phenol depending on market conditions.

There are many methods for dehydrogenating various compounds into phenol. For example, U.S. Pat. No. 4,933,507 discloses that phenol can be produced by dehydrogenating cyclohexenone through a vapor-phase reaction in the presence of hydrogen using a solid-phase catalyst having platinum and alkali metal carried on a support. The catalyst support proposed in the '507 patent is silica, silica-alumina or alumina.

In an article entitled “Performance of activity test on supported Pd catalysts for dehydrogenation of cyclohexanone to phenol (effect of supports on activity)”, Ibaraki Kogyo Koto Senmon Gakko Kenkyu Iho (1995), 30, 39-46 Saito et al. report on the activity and selectivity of various metal oxide (Al₂O₃, TiO₂, ZrO₂, MgO)-supported Pd (1%) catalysts in the dehydrogenation of cyclohexanone to phenol.

More recently, carbon nanotubes have been investigated as supports for dehydrogenation catalysts including cyclohexanone dehydrogenation catalysts. For example, U.S. Published Patent Application No. 2006/0137817 discloses a macroscopic rigid porous carbon structure which comprises intertwined, interconnected single walled carbon nanotubes, said rigid porous carbon structure having a surface area greater than about 800 m²/gm, being substantially free of micropores and having a crush strength greater than about 5 lb/in². In Table IIA, the '817 application lists potential catalytic uses of the porous carbon structure including as a support for a Pt catalysts for the dehydrogenation of cyclohexanone to phenol.

According to the present invention, it has now been found that a catalyst employing a mesoporous, crystalline material, such as MCM-41, exhibits improved properties in the dehydrogenation of cyclohexanone to phenol.

U.S. Pat. No. 7,285,512 discloses a catalyst and process for selectively hydrodesulfurizing naphtha feedstreams using a catalyst comprising at least one hydrodesulfurizing metal supported on a low acidity, ordered mesoporous support material, such as MCM-41.

### SUMMARY

In one aspect, the invention resides in a process for the dehydrogenation of at least one dehydrogenatable hydrocarbon, the process comprising contacting a feed comprising the at least one dehydrogenatable hydrocarbon with a catalyst comprising an inorganic, crystalline, mesoporous support material and a dehydrogenation component under dehydrogenation conditions effective to convert at least part of the at least a portion of the at least one dehydrogenatable hydrocarbon in said feed.

Conveniently, the at least one dehydrogenatable hydrocarbon is an alicyclic compound such as cyclohexane and cyclohexanone wherein at least a portion of the dehydrogenatable hydrocarbon is converted into an aromatic compound such as benzene and phenol.

Conveniently, the at least one dehydrogenatable hydrocarbon is cyclohexanone wherein at least a portion of the cyclohexanone is converted into phenol.

Conveniently, the at least one dehydrogenatable hydrocarbon is cyclohexane wherein at least a portion of the cyclohexane is converted into benzene.

Conveniently, the support material exhibits an X-ray diffraction pattern, after calcination, with at least one peak at a position greater than about 18 Angstrom Units d-spacing with a relative intensity of 100, and has a benzene adsorption capacity of greater than about 15 grams benzene per 100 grams of the anhydrous support material at 50 torr (6.7 kPa) and 25° C. In one embodiment, the support material comprises MCM-41.

Conveniently, the support material comprises a silicate or aluminosilicate having a silica to alumina molar ratio of at least 100, such as at least 500.

Conveniently, the dehydrogenation component comprises at least one metal component selected from Groups 6 to 10 of the Periodic Table of Elements, such as platinum and palladium.

Conveniently, the catalyst further contains an inorganic base component, such as a potassium compound.

Conveniently, the dehydrogenation conditions include a temperature of about 250° C. to about 500° C., a pressure of about atmospheric to about 500 psig (100 to 3550 kPa), a weight hourly space velocity of about 0.2 to about 50 hr⁻¹, and a hydrogen to cyclohexanone-containing feed molar ratio of about 0 to about 20.

In a further aspect, the invention resides in a process for producing phenol from benzene, the process comprising:

(a) contacting benzene and hydrogen with a catalyst under hydroalkylation conditions to produce cyclohexylbenzene;
(b) oxidizing at least a portion of the cyclohexylbenzene from (a) to produce cyclohexylbenzene hydroperoxide;
(c) converting at least a portion of the cyclohexylbenzene hydroperoxide from (b) to produce an effluent steam comprising phenol and cyclohexanone; and
(d) contacting at least a portion of said effluent stream with a catalyst comprising an inorganic, crystalline, mesoporous support material and a dehydrogenation component under dehydrogenation conditions effective to convert at least part of the cyclohexanone in said effluent portion into phenol and hydrogen.

### BRIEF DESCRIPTION OF THE DRAWINGS

FIG. 1 shows graphs of cyclohexanone conversion as a function of time-on-stream (TOS) for the 1% Pt/1% K/MCM-41 catalysts of Example 3 wherein “X” represents “conversion.”

FIG. 2 shows graphs of phenol selectivity as a function of time-on-stream (TOS) for the 1% Pt/1% K/MCM-41 catalysts of Example 3 wherein “S” represents “selectivity.”

FIG. 3 shows graphs of cyclohexanone conversion, phenol selectivity and benzene selectivity as a function of time-on-stream (TOS) for the 0.6% Pt/1% K/ZrO₂ catalyst of Comparative Example 1 wherein “X” and “S” represents “conversion” and “selectivity”, respectively.

FIGS. 4 and 5 show graphs of cyclohexanone conversion, phenol selectivity and benzene selectivity as a function of time-on-stream (TOS) for the 1% Pt/Carbon Nanotube (CNT) and 1% Pt/1% K/CNT catalysts of Comparative Example 2 wherein “X” and “S” represents “conversion” and “selectivity”, respectively.

### DETAILED DESCRIPTION OF THE EMBODIMENTS

Described herein is a process for dehydrogenating at least one dehydrogenatable hydrocarbon such as cyclohexanone wherein the dehydrogenation catalyst support comprises an inorganic, crystalline, mesoporous support material. Specifically, this dehydrogenation process can be utilized in a phenol process wherein cyclohexanone is co-produced with phenol by allowing at least a portion of the co-produced cyclohexanone to be converted to additional phenol. In the phenol process wherein cyclohexanone is co-produced, cyclohexylbenzene, generally produced by the catalytic hydroalkylation of benzene, is oxidized to produce cyclohexylbenzene hydroperoxide and then the cyclohexylbenzene hydroperoxide is cleaved to produce an effluent steam comprising phenol and cyclohexanone in substantially equimolar amounts. At least a portion of the effluent is then fed to a dehydrogenation reaction zone, where the effluent stream portion is contacted with a dehydrogenation catalyst so as to convert the cyclohexanone in said effluent portion into additional phenol and into hydrogen, which can be recycled to the benzene hydroalkylation step.

### Dehydrogenation Catalyst and Process

The dehydrogenation process may be used to dehydrogenate any dehydrogenatable hydrocarbon such as an alicyclic compound. “Dehydrogenatable hydrocarbon” refers to all classes of hydrocarbons containing saturated carbon bonds which have the potential for forming one or more unsaturated bonds through the process of dehydrogenation. “Alicyclic compounds” refers to saturated or unsaturated non-aromatic hydrocarbon ring systems containing from three to twenty ring carbon atoms wherein the hydrocarbon ring system may also have a side-chain or a functional group attached directly to or bound within the ring. Examples of alicyclic compounds include, without limitation, cyclopropane, cyclopentane, methyl cyclopentane, cyclobutane, cyclopentene, cyclodecane, cyclohexane, methylcyclohexane, cyclododecane, and six carbon ring alicyclic compounds such as cyclohexane. Other examples of alicyclic compounds include without limitation alicyclic ketones such as cyclohexanone and alicyclic alcohols such as cyclohexanol.

In one embodiment, at least a portion of the six carbon ring alicyclic compounds are dehydrogenated (or converted) to aromatic compounds such as benzene and phenol. For example, at least a portion of cyclohexanone may be dehydrogenated to phenol and at least a portion of cyclohexane may be dehydrogenated to benzene.

In another embodiment, at least a portion of the alicyclic compounds are (i) dehydrogenated to unsaturated compounds, (ii) rearranged to form other alicyclic compounds or (iii) fragment to lighter hydrocarbons.

The catalyst support employed in the dehydrogenation reaction comprises an inorganic, crystalline, mesoporous support material and a dehydrogenation component. In this respect, the term “mesoporous” is used herein to refer to porous material having a maximum perpendicular cross-section pore dimension of at least about 13 Angstroms, and generally within the range of from about 13 Angstroms to about 200 Angstroms. The catalyst support may also be non-layered wherein non-layered is herein defined as non-lamellar.

In layered (i.e., lamellar) materials, the interatomic bonding in two directions of the crystalline lattice is substantially different from that in the third direction, resulting in a structure that contains cohesive units resembling sheets. Usually, the bonding between the atoms within these sheets is highly covalent, while adjacent layers are held together by ionic forces or van der Waals interactions. These latter forces can frequently be neutralized by relatively modest chemical means, while the bonding between atoms within the layers remains intact and unaffected.

In one embodiment, the mesoporous support material exhibits an X-ray diffraction pattern, after calcination, with at least one peak at a position greater than about 18 Angstrom Units d-spacing with a relative intensity of 100, and has a benzene adsorption capacity of greater than about 15 grams benzene per 100 grams of the anhydrous support material at 50 torr (6.7 kPa) and 25° C. One example of such a mesoporous support material is MCM-41, which has a hexagonal arrangement of uniformly-sized pores and is described in U.S. Pat. No. 5,098,684, the entire contents of which are incorporated herein by reference. Other suitable support materials include MCM-48, which has a cubic symmetry and is described in U.S. Pat. No. 5,198,203, and MCM-50, which has a lamellar structure and is described in U.S. Pat. No. 5,304,363. The entire contents of both of these patents are incorporated herein by reference.

In one embodiment, the support material comprises a silicate or aluminosilicate having a silica to alumina molar ratio of at least 100, such as at least 500. In still another embodiment, the support material comprises a silicate or aluminosilicate having a silica to alumina molar ratio of from 100 to 5,000; from 100 to 4,000; from 100 to 3,000; from 100 to 2,000; from 100 to 1,000; from 500 to 5,000; from 500 to 4,000; from 500 to 3,000; or from 500 to 2,000.

Generally, the dehydrogenation component employed in the present catalyst comprises at least one metal component selected from Groups 6 to 10 of the Periodic Table of Elements, such as platinum and palladium. The dehydrogenation component may also comprise any combination or mixture of metal components selected from Groups 6 to 10 of the Periodic Table of Elements. Typically, the dehydrogenation component is present in an amount between about 0.1 and about 10 wt % of the catalyst. The term “metal component” is used herein to include a metal compound that may not be purely the elemental metal, but could, for example, be at least partly in another form, such as an oxide, hydride or sulfide form.

In one embodiment, the catalyst comprises a secondary component comprising at least one metal component selected from Group 1 and Group 2 of the Periodic Table of Elements, such as potassium, cesium, and rubidium wherein said at least one metal component selected from Group 1 and Group 2 of the Periodic Table of Elements is present in an amount of at least 0.1 wt %, at least 0.2 wt %, at least 0.3 wt %, at least 0.4 wt % or at least 0.5 wt %. The second component may also comprise any combination or mixture of metal components selected from Group 1 and Group 2 of the Periodic Table of Elements. Typically, the secondary component is present in an amount between about 0.1 and about 5 wt % of the catalyst, preferably between 0.1 and 3 wt %, more preferably between 0.1 and 2 wt % of the catalyst. In one embodiment, the secondary component is a potassium compound.

The dehydrogenation catalyst is typically prepared by initially treating the support, such as by impregnation, with a liquid composition comprising the dehydrogenation component or a precursor thereof, the optional inorganic base component and at least one organic dispersant dispersed in a liquid carrier, such as water. The organic dispersant is generally selected from an amino alcohol and an amino acid, and typically comprises arginine. Generally, the organic dispersant is present in the liquid composition in an amount between about 1 and about 20 wt % of the liquid composition.

The catalyst may be treated with the dehydrogenation component and the inorganic base component in any sequence or simultaneously wherein the organic dispersant may be used when treating with the dehydrogenation component or the inorganic component or both.

After treatment with the liquid composition, the support is dried to remove the liquid carrier and is then heated in an oxidizing atmosphere, such as air, under conditions to decompose substantially all of said organic dispersant. Suitable conditions for removing the dispersant include a temperature of about 100° C. to about 600° C. for a time of about 0.5 to about 50 hours. The catalyst may then be heated in a reducing atmosphere, such as hydrogen, at a temperature of about 50° C. to about 500° C. for a time of about 0.5 to about 10 hours to reduce the dehydrogenation component.

Suitable conditions for the dehydrogenation step include a temperature of about 250° C. to about 750° C., a pressure of about atmospheric to about 500 psig (100 to 3550 kPa), a weight hourly space velocity of about 0.2 to 50 hr⁻¹, and a hydrogen to cyclohexanone-containing feed molar ratio of about 0 to about 20. Other conditions include a temperature of about 250° C. to about 500° C.

### Production of Cyclohexylbenzene

The cyclohexylbenzene employed in the present process can be produced by any conventional technique, including alkylation of benzene with cyclohexene in the presence of an acid catalyst, such as zeolite beta or an MCM-22 family molecular sieve, or by oxidative coupling of benzene to biphenyl followed by hydrogenation of the biphenyl. However, in practice, the cyclohexylbenzene is generally produced by contacting benzene with hydrogen under hydroalkylation conditions in the presence of a hydroalkylation catalyst whereby the benzene undergoes the following reaction (1) to produce cyclohexylbenzene (CHB):

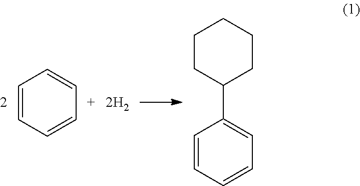

Details of such a process for producing cyclohexylbenzene can be found in paragraphs [0027] through [0038] of WO 2009/131769, the disclosure of which is hereby incorporated by reference.

### Cyclohexylbenzene Oxidation

In order to convert the cyclohexylbenzene into phenol and cyclohexanone, the cyclohexylbenzene is initially oxidized to the corresponding hydroperoxide. This is accomplished by introducing an oxygen-containing gas, such as air, into a liquid phase containing the cyclohexylbenzene. Unlike cumene, atmospheric air oxidation of cyclohexylbenzene in the absence of a catalyst is very slow and hence the oxidation is normally conducted in the presence of a catalyst.

Details of such a process for producing cyclohexylbenzene can be found in paragraphs [0048] through [0055] of WO 2009/131769, the disclosure of which is hereby incorporated by reference.

### Hydroperoxide Cleavage

The final reactive step in the conversion of the cyclohexylbenzene into phenol and cyclohexanone involves cleavage of the cyclohexylbenzene hydroperoxide, which is conveniently effected by contacting the hydroperoxide with a catalyst in the liquid phase at a temperature of about 20° C. to about 150° C., such as about 40° C. to about 120° C., a pressure of about 50 to about 2,500 kPa, such as about 100 to about 1000 kPa. The cyclohexylbenzene hydroperoxide is preferably diluted in an organic solvent inert to the cleavage reaction, such as methyl ethyl ketone, cyclohexanone, phenol or cyclohexylbenzene, to assist in heat removal. The cleavage reaction is conveniently conducted in a catalytic distillation unit.

Details of such a process for hydroperoxide cleavage can be found in paragraphs [0056] through [0075] of WO 2009/131769, the disclosure of which is hereby incorporated by reference.

### Treatment of Cleavage Effluent

The effluent from the cleavage reaction comprises phenol and cyclohexanone in substantially equimolar amounts. The present process provides an advantageous route to increasing the amount of phenol produced from the original benzene feed by contacting at least a portion of the cleavage effluent with a dehydrogenation catalyst so as to convert some or all of the cyclohexanone in the effluent into additional phenol according to the reaction (2):

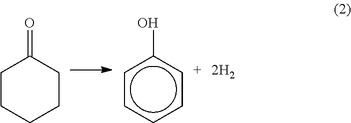

In one embodiment, the dehydrogenation catalyst and process described herein may be used in reaction (2).

Cyclohexanone and phenol produce an azeotropic mixture composed of 28 wt % cyclohexanone and 72 wt % phenol, so that any attempt to separate the effluent from the cyclohexylbenzene hydroperoxide cleavage step by simple distillation results in this azeotropic mixture. However, the efficiency of the separation can be enhanced by conducting the distillation under at least partial vacuum, typically at below 101 kPa. Moreover, extractive distillation processes are known for separating cyclohexanone and phenol, see for example, U.S. Pat. Nos. 4,021,490; 4,019,965; 4,115,207; 4,115,204; 4,115,206; 4,201,632; 4,230,638; 4,167,456; 4,115,205; and 4,016,049. Nevertheless, phenol/cyclohexanone separation remains a costly process, so that in one embodiment, the feed to the dehydrogenation step has the same composition as the cleavage effluent, thereby avoiding the need for an initial expensive separation step. Depending on the efficiency of the cyclohexanone dehydrogenation, the final product may contain substantially all phenol, thereby at least reducing the problem of separating the phenol from the cleavage effluent.

In another embodiment, the cleavage effluent is subjected to one or more separation processes to recover or remove one or more components of the effluent prior to dehydrogenation. In particular, the cleavage effluent is conveniently subjected to at least a first separation step to recover some or all of the phenol from the effluent, typically so that the effluent stream fed to said dehydrogenation reaction contains less than 50 wt %, for example less than 30 wt %, such as less than 1 wt %, phenol. The first separation step is conveniently effected by vacuum distillation and the same, or additional vacuum distillation steps, can be used to remove components boiling below 155° C. (as measured at 101 kPa), such as benzene and cyclohexene, and/or components boiling above 185° C. (as measured at 101 kPa), such as 2-phenyl phenol and diphenyl ether, prior to feeding the effluent stream to the dehydrogenation reaction.

By employing the present dehydrogenation process, substantially all the cyclohexanone in the cyclohexylbenzene hydroperoxide cleavage effluent can be converted to phenol. In practice, however, depending on market conditions, there is likely to be a significant demand for cyclohexanone product. This can readily be met by using the present process by reliance on the reversible nature of the reaction (2), namely by hydrogenating at least some of the phenol back to cyclohexanone. This can readily be achieved by, for example, contacting the phenol with hydrogen in the presence of a hydrogenation catalyst, such as platinum or palladium, under conditions including a temperature of about 20° C. to about 250° C., a pressure of about 101 kPa to about 10000 kPa and a hydrogen to phenol molar ratio of about 1:1 to about 100:1.

The invention will now be more particularly described with reference to the following non-limiting Examples and the accompanying drawings.

In the Examples, catalytic testing was conducted using catalyst particles having a size between 30 to 40 mesh produced by pressing the catalytic materials described below into thin disks using a hydraulic press at a pressure of about 5 ton and then crushing and sieving the disks.

600 mg of each pelletized catalyst was mixed with 3.5 g of about 40 mesh quartz chips, and the mixture was packed into a ⅜ inch (9.5 mm) internal diameter stainless steel downflow reactor. A thermocouple was inserted from the bottom of the reactor into the center of the roughly 5″ (12.7 cm) catalyst bed for measuring catalyst bed temperature.

Prior to the introduction of cyclohexanone feed, the catalyst was pretreated in 72 sccm H₂ at 100 psig (760 kPa) by ramping the reactor temperature from room temperature to 425° C. at 2° C./min and then holding the reactor temperature at 425° C. for 2 hrs under the same H₂ flow and pressure to allow for reduction of the supported catalyst prior to testing.

Cyclohexanone feed was delivered at 9.5 ml/hr using an ISCO pump. Cyclohexanone feed was vaporized prior to mixing with 72 sccm of H₂. The reaction was typically run at 425° C. and 100 psig (760 kPa) total reactor pressure, so the cyclohexanone partial pressure was 37 psia (255 kPa). The weight hourly space velocity (WHSV) worked out to be about 15 hr⁻¹. The H₂/cyclohexanone molar ratio of the feed was 2 to 1.

The effluent from the reactor was sampled using a Valco sampling valve, and the sample was sent to an on-line GC equipped with a FID for analysis. All the hydrocarbons were analyzed and the results were normalized. H₂ was not included in the analysis. Conversion was calculated based on the concentration of cyclohexanone in the effluent. Other components which was typically present in the effluent, was counted as unreacted feed. All the concentratrations shown here are in wt %.

### Example 1

### Preparation of Small Pore (˜20 Å) Si-MCM-41 with SiO₂/Al₂O₃ of about 800/1 Molar Ratio

A mixture was prepared from 788 grams of water, 158 grams of n-decyltrimethylammonium bromide solution, 235 grams of 35 wt % tetraethylammonium hydroxide (TEAOH) solution, and 221 grams of Ultrasil silica. The mixture was reacted at 240° F. (116° C.) in a 2-liter autoclave with stirring at 90 RPM for 36 hours. The product was filtered, washed with deionized (DI) water, followed by drying at 250° F. (120° C.) and calcination at 1000° F. (540° C.) for 6 hours. The XRD pattern of the as-synthesized material showed the typical pure phase of MCM-41 topology. The SEM of the as-synthesized material showed that the material was composed of agglomerates of small crystals. The resulting Si-MCM-41 crystals had a SiO₂/Al₂O₃ molar ratio of about 800/1, a surface area of about 1,100 m²/g and a pore size of about 20 Å. The sample was denoted as MCM-41(20).

### Example 2

### Preparation of Large Pore (˜60 Å) Si-MCM-41 with SiO₂/Al₂O₃ of about 800/1

A mixture was prepared from 737 grams of water, 306 grams of Arquad 16/29 solution (a commercially available surfactant from Akzo Nobel), 56 g of 50 wt % NaOH solution, 198 g of Mesitylene 97 wt % solution, and 182 grams of Ultrasil silica. The mixture was reacted at 240° F. (116° C.) in a 2-liter autoclave with stirring at 90 RPM for 36 hours. The product was filtered, washed with deionized (DI) water, followed by drying at 250° F. (120° C.) and calcination at 1000° F. (540° C.) for 6 hrs. The XRD pattern of the as-synthesized material showed the typical pure phase of MCM-41 topology. The SEM of the as-synthesized material showed that the material was composed of agglomerates of small crystals. The resulting Si-MCM-41 crystals had a SiO₂/Al₂O₃ molar ratio of about 800/1, a surface area of about 800 m²/g and a pore size of about 60 Å. The sample was denoted as MCM-41(60).

### Example 3

### Preparation and Testing of 1% Pt/1%/K/MCM-41(20) and 1% Pt/1%/K/MCM-41(60)

MCM-41(20) and MCM-41(60) were calcined at 1000° F. (540° C.) in air for 2 hours to obtain Na-form calcined crystals. Then 1 wt % K was impregnated onto the calcined materials with 0.5N KOH solution via incipient wetness followed by drying at 250° F. (120° C.) and calcination in full air at 1000° F. (540° C.) for 2 hours. 1 wt % Pt was then impregnated onto the K/MCM-41 samples with platinum tetraamine hydroxide solution via incipient wetness followed by drying at 250° F. (120° C.) and calcination in full air at 680° F. (360° C.) for 2 hours. The finished samples were denoted as 1% Pt/1%/K/MCM-41(20) and 1% Pt/1%/K/MCM-41(60).

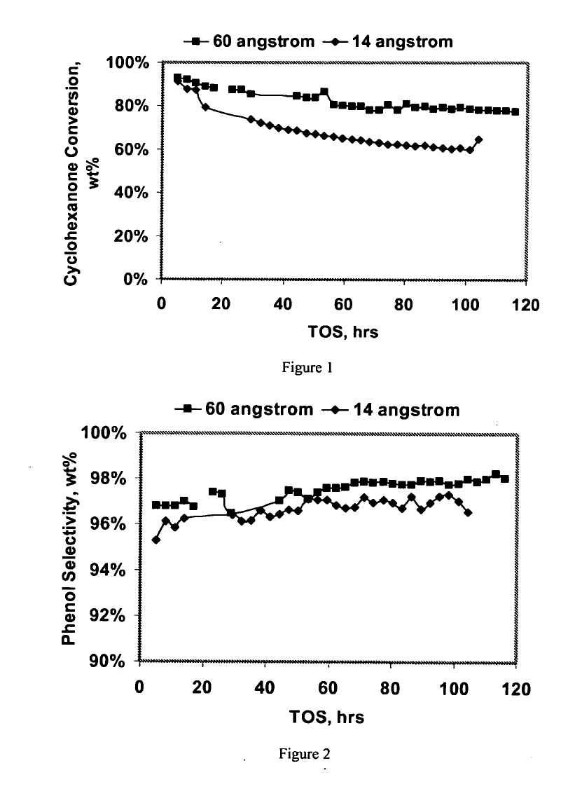

The resultant catalysts were tested for the dehydrogenation of cyclohexanone according to the testing regime outlined above and the results are shown in FIGS. 1 and 2. It will be seen that both the 1% Pt/1%/K/MCM-41(20) and 1% Pt/1%/K/MCM-41(60) samples were very effective catalysts giving an initial cyclohexanone conversion of over 90% and a phenol selectivity in excess of 95%.

### Comparative Example 1

### Preparation and Testing of 0.6% Pt/1% K/ZrO₂

A low surface area, <20 m²/g, zirconia powder was calcined at 540° C. for 4 hours in air before the Pt impregnation. Then 0.6 wt % of Pt was supported on this calcined zirconia by the incipient-wetness method using a solution of platinum tetraamine nitrate, followed by drying and air calcination at 680° F. (360° C.) for 2 hrs. The sample was denoted at 0.6% Pt/ZrO2. 1% of K was impregnated onto 0.6%/ZrO₂ sample by wet impregnation using KOH solution. The sample was dried followed by air calcination at 680° F. (360° C.) for 2 hours. The finished sample was denoted as 0.6% Pt/1% K/ZrO₂.

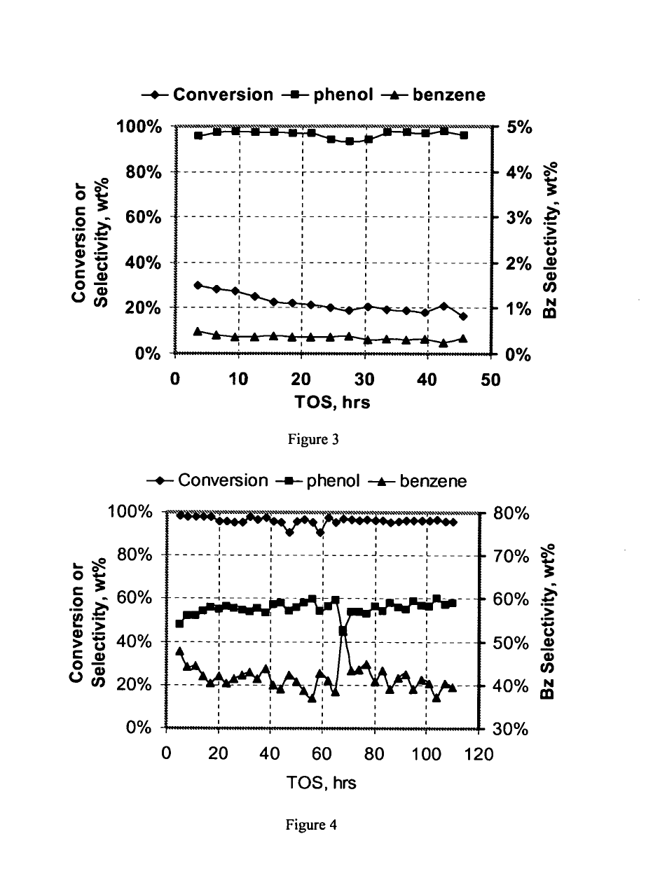

The resultant catalyst was tested for the dehydrogenation of cyclohexanone according to the testing regime outlined above and the results are shown in FIG. 3. It will be seen that, although the selectivity for phenol is over 95 wt % while the selectivity for benzene is very low at below 0.5 wt %, the conversion for cyclohexanone is relatively low (about 20 wt %). In contrast, the MCM-41 supported catalysts of Example 3 are significantly more active than the ZrO₂ catalyst of Comparative Example 1.

### Comparative Example 2

### Preparation and Testing of 1% Pt/CNT and 1% Pt/1% K/CNT

1 wt % of Pt was impregnated onto a carbon nanotube sample by wet impregnation using 3.55 wt % of platinum tetraamine nitrate solution. The sample was dried at 120° C. for 2 hrs. The dried sample was denoted as 1% Pt/CNT. 1 wt % of K was impregnated onto part of 1% Pt/CNT sample by wet impregnation using K₂CO₃ solution. The sample was then dried at 120° C. for 2 hours. The sample was denoted as 1% Pt/1% K/CNT.

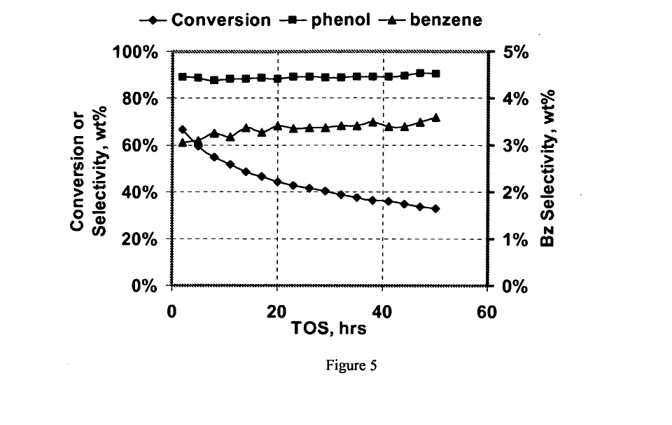

The resultant catalysts were tested for the dehydrogenation of cyclohexanone according to the testing regime outlined above and the results are shown in FIG. 4 (1% Pt/CNT) and FIG. 5 (1% Pt/1% K/CNT). It will be seen that, although 1% Pt/CNT is much more active than 1% Pt/1% K/CNT, the latter is much more selective for phenol than the former (i.e., about 90% phenol selectivity for 1% Pt/1% K/CNT as compared with about 55% selectivity for phenol for 1% Pt/CNT).

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

In another embodiment, this disclosure relates to:

1. A process for the dehydrogenation of at least one dehydrogenatable hydrocarbon, the process comprising contacting a feed comprising the at least one dehydrogenatable hydrocarbon with a catalyst comprising an inorganic, crystalline, mesoporous support material and a dehydrogenation component under dehydrogenation conditions effective to convert at least part of the at least one dehydrogenatable hydrocarbon in the feed.
2. The process of embodiment 1, wherein the support material exhibits an X-ray diffraction pattern, after calcination, with at least one peak at a position greater than about 18 Angstrom Units d-spacing with a relative intensity of 100, and has a benzene adsorption capacity of greater than about 15 grams benzene per 100 grams of the anhydrous support material at 50 torr (6.7 kPa) and 25° C.
3. The process of embodiment 1, wherein the support material is non-layered.
4. The process of embodiment 1, wherein the support material comprises MCM-41.
5. The process of embodiment 1, wherein the support material comprises silica and alumina and wherein the support material has a silica to alumina molar ratio of at least 100.
6. The process of embodiment 1, wherein the support material comprises silica and alumina and wherein the support material has a silica to alumina molar ratio of at least 500.
7. The process of embodiment 1, wherein the at least one dehydrogenatable hydrocarbon is an alicyclic compound.
8. The process of embodiment 1, wherein the at least one dehydrogenatable hydrocarbon is cyclohexanone.
9. The process of embodiment 1, wherein the dehydrogenation component comprises at least one metal component selected from Groups 6 to 10 of the Periodic Table of Elements.
10. The process of embodiment 1, wherein the dehydrogenation component comprises at least one metal component selected from platinum and palladium.
11. The process of embodiment 1, wherein the catalyst further contains an inorganic base component.
12. The process of embodiment 11, wherein the inorganic base component comprises an alkali or alkaline earth metal compound.
13. The process of embodiment 11, wherein the inorganic base component comprises a potassium compound.
14. The process of embodiment 1, wherein the dehydrogenation conditions include a temperature of about 250° C. to about 500° C., a pressure of about atmospheric to about 500 psig (100 to 3550 kPa), a weight hourly space velocity of about 0.2 to about 50 and a hydrogen to cyclohexanone-containing feed molar ratio of about 2 to about 20.
15. A process for producing phenol from benzene, the process comprising:
(a) contacting benzene and hydrogen with a catalyst under hydroalkylation conditions to produce cyclohexylbenzene;
(b) oxidizing cyclohexylbenzene to produce cyclohexylbenzene hydroperoxide;
(c) converting at least a portion of the cyclohexylbenzene hydroperoxide from oxidizing (b) to produce an effluent steam comprising phenol and cyclohexanone; and
(d) contacting at least a portion of the effluent stream with a catalyst comprising an inorganic, crystalline, mesoporous support material and a dehydrogenation component under dehydrogenation conditions effective to convert at least part of the cyclohexanone in the effluent portion into phenol and hydrogen.
16. The process of embodiment 15, wherein the support material exhibits an X-ray diffraction pattern, after calcination, with at least one peak at a position greater than about 18 Angstrom Units d-spacing with a relative intensity of 100, and has a benzene adsorption capacity of greater than about 15 grams benzene per 100 grams of the anhydrous support material at 50 torr (6.7 kPa) and 25° C.
17. The process of embodiment 15, wherein the support material is non-layered.
18. The process of embodiment 15, wherein the support material comprises MCM-41.
19. The process of embodiment 15, wherein the support material comprises an aluminosilicate having a silica to alumina molar ratio of at least 100.
20. The process of embodiment 15, wherein the support material comprises an aluminosilicate having a silica to alumina molar ratio of at least 500.
21. The process of embodiment 15, wherein the dehydrogenation component comprises at least one metal selected from Groups 6 to 10 of the Periodic Table of Elements and compounds and mixtures thereof.
22. The process of embodiment 15, wherein the dehydrogenation component comprises platinum, palladium and compounds and mixtures thereof.
23. The process of embodiment 15, wherein the catalyst further contains an inorganic base component.
24. The process of embodiment 21, wherein the inorganic base component comprises an alkali or alkaline earth metal compound.
25. The process of embodiment 21, wherein the inorganic base component comprises a potassium compound.
26. The process of embodiment 15, wherein the dehydrogenation conditions include a temperature of about 250° C. to about 500° C., a pressure of about atmospheric to about 500 psig (100 to 3550 kPa), a weight hourly space velocity of about 0.2 to 50 hr⁻¹, and a hydrogen to cyclohexanone-containing feed molar ratio of about 0 to about 20.
27. The process of embodiment 15, and further comprising:
(e) recycling at least part of the hydrogen produced in the contacting (d) to the contacting (a).

What is claimed is:

1. A process for the dehydrogenation of at least one of cyclohexane and cyclohexanone, the process comprising contacting a feed comprising the at least one of cyclohexane and cyclohexanone, with a catalyst comprising (i) an inorganic, crystalline, mesoporous support material; and (ii) a dehydrogenation component comprising platinum or palladium under dehydrogenation conditions comprising a temperature of 250° C. to 750° C., a pressure of atmospheric to 500 psi, gauge (100 to 3550 kPa) to convert at least part of the at least one of cyclohexane and cyclohexanone in the feed.

2. The process of claim 1, wherein the support material exhibits an X-ray diffraction pattern, after calcination, with at least one peak at a position greater than about 18 Angstrom Units d-spacing with a relative intensity of 100, and has a benzene adsorption capacity of greater than about 15 grams benzene per 100 grams of the anhydrous support material at 50 torr (6.7 kPa) and 25° C.

3. The process of claim 1, wherein the support material is non-layered.

4. The process of claim 1, wherein the support material comprises MCM-41.

5. The process of claim 1, wherein the support material comprises silica and alumina and wherein the support material has a silica to alumina molar ratio of at least 100.

6. The process of claim 1, wherein the support material comprises silica and alumina and wherein the support material has a silica to alumina molar ratio of at least 500.

7.-10. (canceled)

11. The process of claim 1, wherein the catalyst further contains an inorganic base component, preferably an alkali or alkaline earth metal compound, and more preferably a potassium compound.

12. The process of claim 1, wherein the dehydrogenation conditions further include a weight hourly space velocity of 0.2 to 50 hr⁻¹, and a hydrogen to cyclohexanone-containing feed molar ratio of 2 to 20.

13. The process of claim 1, the process and further comprising:
(a) contacting benzene and hydrogen with a catalyst under hydroalkylation conditions to produce cyclohexylbenzene;
(b) oxidizing cyclohexylbenzene to produce cyclohexylbenzene hydroperoxide;
(c) converting at least a portion of the cyclohexylbenzene hydroperoxide from oxidizing (b) to produce an effluent steam comprising phenol and cyclohexanone; and
(d) feeding at least portion of said effluent stream to said contacting (i).

14. The process of claim 13, and further comprising:
(e) recycling at least part of the hydrogen produced in the contacting (d) to the contacting (a).
